(19) United States
(12) Patent Application Publication
(10) Pub. No.: US 2021/0093674 A1
(43) Pub. Date: Apr. 1, 2021

(54) Cell Composition, Method of Production and its Use in Corneal Diseases

(71) Applicants: Hyderabad Eye Research Foundation, Hyderabad, Telangana (IN); Hyderabad Eye Institute, Hyderabad, Telangana (IN)

(72) Inventors: Sayan Basu, Hyderabad, Telangana (IN); Vivek Singh, Hyderabad, Telangana (IN)

(21) Appl. No.: 16/620,187

(22) PCT Filed: Jun. 9, 2018
(86) PCT No.: PCT/IB2018/054183

§ 371 Date: Dec. 6, 2019

(30) Foreign Application Priority Data
Jun. 9, 2017 (IN) 201741020290

(51) Int. Cl. A61K 35/30; A61K 38/36; A61K 38/48; A61P 27/02

(52) U.S. Cl. CPC A61K 35/30; A61P 27/02; A61K 38/4833; A61K 38/363

(57) ABSTRACT

The present invention provides cell compositions, methods of production and its uses in corneal diseases. The invention discloses cell compositions and multilayer cell compositions comprising limbal epithelial cells and limbal stromal cells. The inventions are highly efficacious and represents an advancement over the existing therapeutic approaches in treatment or prevention of corneal diseases. The invention also discloses methods for preparing the compositions, methods of treatment and the uses of the composition in preventing and treating corneal diseases.

### CROSS REFERENCE TO RELATED APPLICATIONS AND CLAIM OF PRIORITY

The present application claims priority from Indian Patent Application No. 201741020290 filed on Jun. 9, 2017, the entire contents of which are hereby incorporated by reference.

### FIELD OF INVENTION

The present invention pertains to the field of pharmaceutical compositions. More particularly, the invention relates to pharmaceutical compositions comprising limbal epithelial cell and limbal stromal cell, method for preparing the composition and its use in preventing and treating corneal diseases.

### BACKGROUND OF THE INVENTION

The cornea is the transparent covering and the main refractive element of the eye. It is responsible for transmission of light to the retina. The human cornea is composed of three primary layers, an outermost epithelium layer, a middle stroma containing keratocytes and an innermost single layer of endothelial cells.

Corneal diseases continue to be one of the leading causes of blindness. The diseases lead to loss of corneal transparency and subsequently deteriorates the vision. There are a wide variety of infectious and inflammatory eye diseases that cause corneal scarring and may result in total blindness.

The most widely accepted treatments for corneal blindness includes replacement of damaged or diseased cornea with a healthy donated cornea or corneal tissue, also known as keratoplasty. When the entire cornea is replaced, the process is known as penetrating keratoplasty and when only part of the cornea is replaced the process is known as lamellar keratoplasty.

Unfortunately, the approach of keratoplasty suffers from several shortcomings due to the following reasons: —
The supply of donor tissue is substantially less than the demand for transplantation that has resulted in a large number of untreated patients worldwide.
Donor cornea is often rejected in a large proportion of patients due to reasons such as autoimmunity, chemical burns, and infections.
Survival rate of corneal grafts decreases over time.
More than 50% keratoplasty is done in cases with fair to poor prognosis.

Most corneal blindness in India and the rest of the developing world is due to corneal epithelial and stromal pathologies rather than endothelial diseases. Whenever the cornea is damaged it heals by both epithelial overgrowth and stromal scarring. These natural healing responses lead to deterioration in corneal clarity and impairment of normal visual function. Recent advances in regenerative medicine have opened up the possibility of using ocular stem cells for treatment of corneal pathologies. Epithelial stem cell transplantation, both autologous and allogeneic have been in clinical practice for the last two decades. However, restoration of the epithelium alone does not improve vision since the stromal component is not addressed. Thus, epithelial stem cell transplantation often fails in terms of achieving efficacy in terms of critical parameters such as corneal clarity and visual recovery.

For the first time, the inventors have identified the issues with the therapeutic modalities used till date and has addressed the same by employing a unique approach by preparation of a cell composition comprising epithelial stem cells and stromal stem cells. The inventors have also devised a multilayer composition comprising stromal stem cells in the first layer and epithelial stem cells in the second layer. The inventors have identified that the composition with cell combination as well as multilayer compositions exhibit synergistic effects and can be used for successfully treating various corneal pathologies.

The multilayer composition of the present invention employs a biomimetic approach and mimics the natural corneal physiology. Further, the inventors have identified that the ratio of stromal stem cells and epithelial stem cells in the cell compositions can be customized according to the pathological conditions as per the extent of damage to corneal layers for successful therapeutic intervention.

Thus, the present invention thus contemplates to overcome the problems of the prior art to solve a long-standing problem of providing a pharmaceutical composition with improved efficacious effects. Further, the approach used for development of this invention would make the treatment of corneal diseases more accessible and affordable to the world's visually impaired more specifically for people in low and middle-income countries.

### SUMMARY OF THE INVENTION

### Technical Problem

The technical problem to be solved in this invention is providing a pharmaceutical composition which provides enhanced corneal clarity and visual recovery, as compared to limbal derived epithelial stem cell therapy.

### Solution to the Problem

The problem has been solved by development of cell compositions comprising limbal derived stromal cells and limbal derived epithelial cells. A synergistic and efficacious multilayer cell composition has also been developed, which comprises:
(a) a first layer comprising a plurality of limbal derived stromal cells and thrombin; and
(b) a second layer comprising a plurality of limbal derived epithelial cells and fibrinogen.

The compositions provide higher efficacy than epithelial stem cells therapy and consequently provides high corneal clarity and faster visual recovery.

### Overview of the Invention

In one aspect, the invention provides a cell composition a plurality of limbal derived stromal cells and a plurality of limbal derived epithelial cells. The ratio of limbal derived stromal cells to limbal derived stromal cells can be customized according to the pathological requirements as per the extent of damage to corneal layers. Preferably, the ratio ranges between 1:5 to 5:1. Further, the concentration of limbal derived stromal cells and limbal derived epithelial cells ranges between 4000 to 5000 cells/μL.

In another aspect, the invention provides a multilayer cell composition comprising: (a) a first layer comprising a plurality of limbal derived stromal cells and thrombin; and (b) a second layer comprising a plurality of limbal derived epithelial cells and fibrinogen. The ratio of limbal derived stromal cells to limbal derived stromal cells can be customized according to the pathological requirements as per the extent of damage to corneal layers. Preferably, the ratio ranges between 1:5 to 5:1. Further, the concentration of limbal derived stromal cells and limbal derived epithelial cells ranges between 4000 to 5000 cells/μL.

In another aspect, the invention provides a method for preparing the multilayer cell composition by depositing a first layer comprising a plurality of limbal derived stromal cells and thrombin. Further, a second layer comprising a plurality of limbal derived epithelial cells and fibrinogen is deposited over the first layer.

In yet another aspect, the invention provides a system for delivering a limbal epithelial tissue producing composition to a treatment site in an individual comprising a delivery system, wherein the delivery system comprises the injectable multilayer cell composition.

In another aspect, the invention provides a method of treating a disease or disorder of the eye in a patient by layering the cell composition on the corneal or ocular surface of an individual.

In a further aspect, the invention provides for the use of the multilayer composition for treatment of a disease or disorder of the eye in a patient.

### BRIEF DESCRIPTION OF THE DRAWINGS

FIG. 1 depicts the pure population of stromal cells (P₃) using the monolayer culture.

FIG. 2 depicts limbal derived mesenchymal/stromal stem cells showing expression of CK19, PAX6, Wnt 7a and Col III, obtained after monolayer culture.

FIG. 3 depicts the results of epithelial stem cell therapy on a dysfunctional epithelium of a cornea.

FIG. 4 depicts the results of the application of the cell composition comprising a mixture of epithelial and stromal stem cells on a dysfunctional epithelium of a cornea.

FIG. 5 depicts the results of the application of the multilayer composition on a dysfunctional epithelium of a cornea.

FIG. 6 depicts the comparison of the results of epithelial stem cell therapy, application of cell composition and the application of the multilayer composition on the epithelial clarity of a dysfunctional cornea.

FIG. 7 depicts the comparison of the results of epithelial stem cell therapy, application of cell composition and the application of the multilayer composition on the stromal clarity of a dysfunctional cornea.

FIG. 8 depicts the comparison of the results of epithelial stem cell therapy, application of cell composition and the application of the multilayer composition on the total corneal clarity of a dysfunctional cornea.

FIG. 9 depicts the comparison of the results of epithelial stem cell therapy, application of cell composition and the application of the multilayer composition on the visual recovery of a dysfunctional cornea.

### DEFINITIONS

Unless defined otherwise, all technical and scientific terms used herein have the same meaning as commonly understood by one of ordinary skill in the art to which the methods belong. Although any compositions and methods similar or equivalent to those described herein can also be used in the practice or testing of the methods and compositions, representative illustrative methods and compositions are now described.

Where a range of values is provided, it is understood that each intervening value between the upper and lower limit of that range and any other stated or intervening value in that stated range, is encompassed within by the methods and compositions. The upper and lower limits of these smaller ranges may independently be included in the smaller ranges and are also encompassed within by the methods and compositions, subject to any specifically excluded limit in the stated range. Where the stated range includes one or both of the limits, ranges excluding either or both of those included limits are also included in the methods and compositions.

It is appreciated that certain features of the methods, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other embodiments without departing from the scope or spirit of the present methods. Any recited method can be carried out in the order of events recited or in any other order that is logically possible.

The term “cell composition” as used herein refers to a pharmaceutical concoction comprising a plurality of limbal derived stromal cells and a plurality of limbal derived epithelial cells. Preferably, the limbal derived stromal cells and limbal derived epithelial cells are present in the ratio from 1:5 to 5:1, and the concentration of cells ranges between 4000 to 5000 cells/μL. The stem cells are autologous or allogenic in nature and is preferably layered on the corneal or ocular surface of an individual.

The term “multilayer cell composition” or “multilayer composition” as used herein refers to a pharmaceutical composition containing two layers, wherein the first layer comprises a plurality of limbal derived stromal cells and thrombin; and the second layer comprises a plurality of limbal derived epithelial cells and fibrinogen. Preferably, the limbal derived stromal cells and limbal derived epithelial cells are present in the ratio from 1:5 to 5:1, and the concentration of cells ranges between 4000 to 5000 cells/μL. The stem cells are autologous or allogenic in nature and is preferably layered on the corneal or ocular surface of an individual.

The term “limbal derived stromal cells” refers to stem cells in the mammalian stroma which displays properties of mesenchymal stem cells, including clonal growth, multipotent differentiation, and expression of an array of mesenchymal stem cell-specific markers.

The term “limbal derived epithelial cells” refers to corneal epithelial stem cells found at the basal layer of limbal epithelium of the cornea.

The term “delivery system” refers to a system capable of delivering cell compositions to a treatment site in an individual. Preferably, the cell compositions can be delivered using an injectable syringe or deposited over the corneal epithelium.

The term “corneal clarity”, as used herein refers to the measurement of corneal light scattering using digital imaging, corneal densitometry or any suitable method known in the art.

The term “subject” or “patient” refers to an animal which can be treated by compositions of the invention. The animal may have, be at risk for, or be believed to have or be at risk for a disease or condition that can be treated by compositions and/or methods of the present invention. Animals that can be treated in accordance with the invention include vertebrates, with mammals such as bovine, canine, equine, feline, ovine, porcine, and primate (including humans and non-human primates) animals being particularly preferred examples.

### DETAILED DESCRIPTION OF THE INVENTION

The present invention discloses cell compositions comprising limbal epithelial stem cells and limbal stromal stem cells. The present disclosure provides composition, methods of producing the composition, methods of treatment and using the composition to regenerate thick, strong and organized tissues in corneal epithelium.

The inventors have found that compositions comprising limbal derived epithelial cells and limbal derived stromal cells have a synergistic effect on the therapeutic efficacy in treatment of a diseases or disorder of the corneal epithelium. Further, the inventors have contemplated a unique approach in preparation of a multilayer composition by providing a first layer comprising stromal stem cells and a second layer comprising epithelial stem cells. The composition generates natural tissue constructs which are highly useful for addressing various corneal pathologies such as Steven Johnson's Syndrome (SJS), Limbal Stem Cell Deficiency (LSCD), persistent epithelial defects, sterile keratitis and necrosis, ocular burns etc. The compositions and methods of the present invention are highly efficacious and represents improved therapeutic approaches as compared to the prior art.

### Effectiveness of the Compositions Over the State of the Art

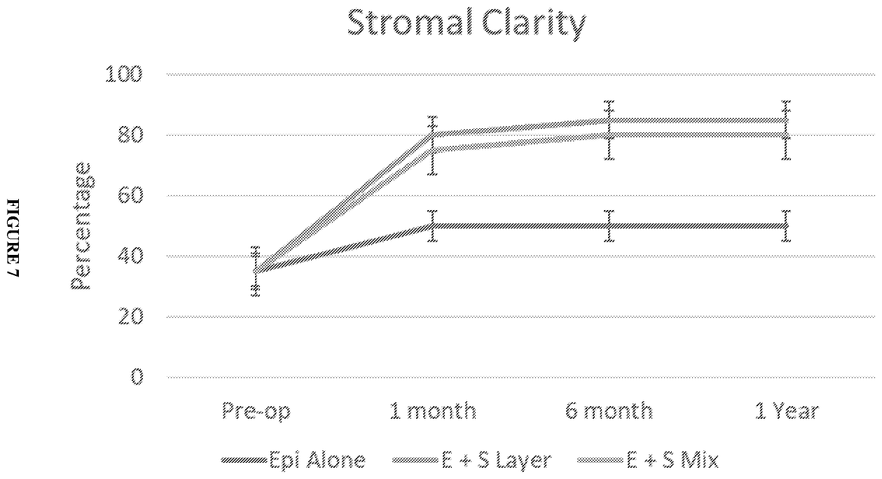

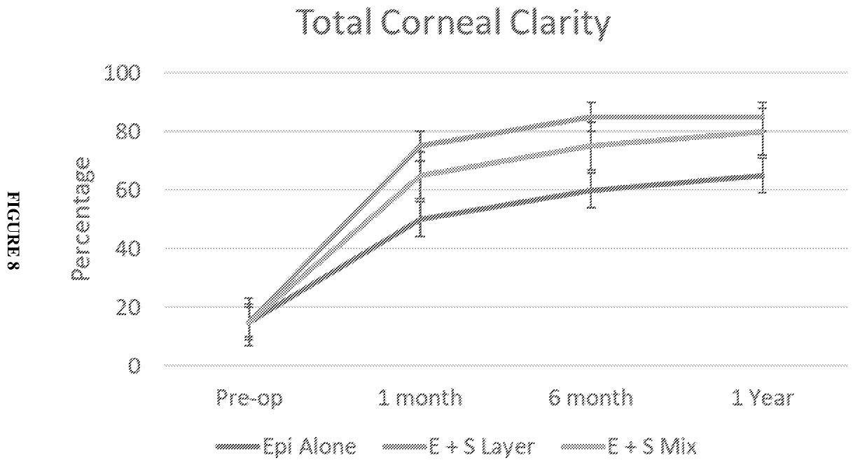

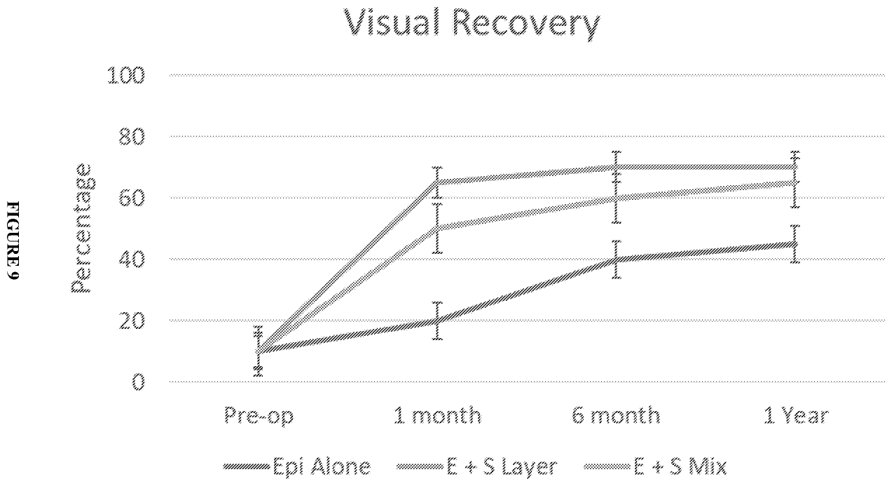

For the first time, the inventors have devised unique compositions which are highly efficacious. The cell composition as well as the multilayer composition are highly efficacious as compared to previous used therapeutic approaches in the following manner:
The compositions provide higher efficacy than epithelial stem cell therapy and consequently provides higher corneal clarity and faster visual recovery, as exhibited in Table 1 (FIG. 6), Table 2 (FIG. 7), Table 3 (FIG. 8) and Table 4 (FIG. 9).
The compositions mimic the natural corneal physiology which results in faster epithelialization of damaged cornea.
The compositions are immunologically safe.
The compositions represent cheaper alternatives than existing surgical interventions.
The therapeutic approach involved in the invention do not require any sophisticated instrument for operation, and as a result can be performed in less-equipped clinics.

Before the compositions and methods of the present disclosure are described in greater detail, it is to be understood that the invention is not limited to particular embodiments and may vary. It is also to be understood that the terminology used herein is for the purpose of describing particular embodiments only, and is not intended to be limiting, since the scope of the compositions and methods will be limited only by the appended claims.

### Cell Composition and Method of Preparing Cell Compositions

The present disclosure provides methods of preparing the cell compositions. The methods involve harvesting and maintaining limbal derived epithelial and limbal derived stromal stem cells under suitable conditions and for a suitable period of time.

In one embodiment, the stem cell cultures free of any mycoplasma contamination is used.

In another embodiment, a cell composition of limbal derived epithelial and limbal derived stromal stem cells is prepared. The cell composition can be customized as per the pathological requirements and the extent of damage to corneal layers. Preferably, the limbal derived stromal stem cells and limbal derived epithelial stem cells are present in the ratio from 1:5 to 5:1.

In one embodiment, limbal derived stromal stem cells and limbal derived epithelial stem cells in the cell composition are present in the ratio from 1:1, 1.25:1, 1.5:1, 1.75:1, 2:1, 2.25:1, 2.5:1, 2.75:1, 3:1, 3.25:1, 3.5:1, 3.75:1, 4:1, 4.25:1, 4.5:1, 4.75:1 and 5:1.

In another embodiment, the limbal derived stromal stem cells and limbal derived epithelial stem cells in the cell composition are present in the ratio from 1:1, 1:1.25, 1:1.5, 1:1.75, 1:2, 1:2.25, 1:2.5, 1:2.75, 1:3, 1:3.25, 1:3.5, 1:3.75, 1:4, 1:4.25, 1:4.5, 1:4.75 and 1:5.

In a further embodiment, the concentration of limbal derived epithelial and stromal stem cells in the cell composition ranges between 4000 to 5000 cells/μL.

### Multilayer Cell Composition and Method of Preparing the Multilayer Cell Composition

In another embodiment, the stromal stem cells are mixed with thrombin, and the epithelial stem cells are mixed with fibrinogen, which are used as the layers of the multilayer composition.

The stromal stem cells mixed with thrombin forms the first layer of the multi-layered composition. The epithelial stem cells mixed with fibrinogen forms the second layer of the multi-layered composition.

The ratio of the stromal stem cells and epithelial stem cells in the multilayer cell composition can be customized as per the pathological requirements and the extent of damage to corneal layers.

In one embodiment the limbal derived stromal stem cells and limbal derived epithelial stem cells in the multilayer cell composition are present in the ratio from 1:5 to 5:1. In another embodiment, the limbal derived stromal stem cells and limbal derived epithelial stem cells in the multilayer cell composition are present in the ratio from 1:1, 1.25:1, 1.5:1, 1.75:1, 2:1, 2.25:1, 2.5:1, 2.75:1, 3:1, 3.25:1, 3.5:1, 3.75:1, 4:1, 4.25:1, 4.5:1, 4.75:1 and 5:1.

In yet another embodiment, the limbal derived stromal stem cells and limbal derived epithelial stem cells in the multilayer cell composition are present in the ratio from 1:1, 1:1.25, 1:1.5, 1:1.75, 1:2, 1:2.25, 1:2.5, 1:2.75, 1:3, 1:3.25, 1:3.5, 1:3.75, 1:4, 1:4.25, 1:4.5, 1:4.75 and 1:5.

In one embodiment, fibrinogen and thrombin in the multilayer cell composition are present in the ratio from 1:5 to 5:1.

In another embodiment, fibrinogen and thrombin in the multilayer cell composition are present in the ratio from 1:1, 1.25:1, 1.5:1, 1.75:1, 2:1, 2.25:1, 2.5:1, 2.75:1, 3:1, 3.25:1, 3.5:1, 3.75:1, 4:1, 4.25:1, 4.5:1, 4.75:1 and 5:1.

In another embodiment, fibrinogen and thrombin in the multilayer cell composition are present in the ratio from 1:1, 1:1.25, 1:1.5, 1:1.75, 1:2, 1:2.25, 1:2.5, 1:2.75, 1:3, 1:3.25, 1:3.5, 1:3.75, 1:4, 1:4.25, 1:4.5, 1:4.75 and 1:5.

In a further embodiment, the concentration of limbal derived epithelial and stromal stem cells in the multilayer cell composition ranges between 4000 to 5000 cells/μL.

For preparation of the multilayer cell composition, a uniform layer of the stromal cell composition (first layer) was first applied on the affected part of the cornea. Thereafter, a uniform layer of the epithelial cell composition (second layer) was applied on the affected part of the cornea.

In an alternate embodiment, stromal stem cells can be mixed with fibrinogen, which forms the first layer of the multi-layered composition. The epithelial stem cells mixed with thrombin, which forms the second layer of the multi-layered composition.

In a further embodiment, the cell composition of limbal derived epithelial and limbal derived stromal stem cells can be applied to the treatment site in an individual. Optionally, thrombin and fibrinogen can be added to the treatment site for scaffolding and tissue formation.

### Systems for Delivery of Cell Compositions

The present disclosure provides a system for delivering the cell compositions to a treatment site in an individual.

A subject system comprises a delivery system that includes an injectable material. The injectable material comprises limbal epithelial or limbal stromal stem cells. The injectable material may further comprise a scaffold or matrix forming component such as fibrinogen or thrombin.

A suitable delivery system can include two syringes, each holding a composition to be admixed. The subject system for delivering a limbal epithelial tissue producing composition to a treatment site in an individual comprises:
(a) a first delivery system comprising a plurality of limbal derived stromal cells and thrombin; and
(b) a second delivery system comprising a plurality of limbal derived epithelial cells and fibrinogen.

In an alternative embodiment, subject system for delivering a limbal epithelial tissue producing composition to a treatment site in an individual comprises:

(a) a first delivery system comprising a plurality of limbal derived stromal cells; and
(b) a second delivery system comprising a plurality of limbal derived epithelial cells.

A suitable delivery system can include a syringe, a syringe and a needle, a syringe and a flexible tubing, capillary cell delivery system, cannula cell delivery systems and the like. A syringe can include a single chamber, or two or more chambers. Preferably, the delivery system includes two or more syringes or a cell delivery system with two chambers.

A scaffold or matrix forming component can include two or more components that, when combined, result in formation of a macromolecular structure. An example is fibrin glue.

In one embodiment, the system comprises limbal epithelial stem cells, limbal stromal stem cells, fibrinogen and thrombin.

The delivery system can include:
(a) a first chamber comprising a plurality of limbal derived stromal cells and thrombin; and
(b) a second chamber comprising a plurality of limbal derived epithelial cells and fibrinogen.

In another embodiment, the invention provides a kit comprising the cell compositions and a delivery system for delivering the cell composition to a treatment site in an individual.

### Treatment of Corneal Epithelial Disorders and Diseases

In one embodiment, the invention provides a method of treating a disease or disorder of the eye in a patient, comprising the step of layering the cell compositions on the corneal or ocular surface of an individual.

In another embodiment, the disease or disorder of the eye is a disease or disorder of the cornea. In yet another embodiment, the disease or disorder of the cornea is a non-healing corneal epithelial defect or persistent corneal epithelial defect.

The disease or disorder may be Steven Johnson's Syndrome, Limbal Stem Cell Deficiency, Persistent Epithelial Defects, Sterile keratitis and necrosis, ocular burns etc.

### EXAMPLES

The following examples particularly describe the manner in which the invention is to be performed. But the embodiments disclosed herein do not limit the scope of the invention in any manner.

### Example 1: Harvesting Epithelial Stem Cells

Therapeutically accepted and serologically tested cadaveric corneas were obtained within four days of collection from the Ramayamma International Eye Bank (L. V. Prasad Eye Institute, Hyderabad, India). The corneas were washed with 1.25 mM penicillin-streptomycin (manufactured by Sigma-Aldrich®) followed by a wash with phosphate buffer saline (manufactured by Sigma-Aldrich®) at pH 7.4 for 3 minutes. It was followed by another wash with phosphate buffer saline.

Iris and endothelial layer were scrapped for visibility. Complete 360° limbal rims were isolated using a surgical blade in buffer saline and minced using a small, curved corneal scissors, in incomplete media (plain DMEM/F-12 media, manufactured by Lonza®).

The tiny limbal tissues pieces were subjected to collagenisation by adding 40 μL of reconstituted Collagenase-IV to the incomplete media (Ser. No. 17/104,019, Thermofisher®) at the rate of 20 μL of Collagenase-IV per mL of incomplete media. The limbal tissue pieces were incubated for 16 hours at 37° C. in 5% CO2 chamber.

Post 16-hour incubation, the enzymatic digestion was stopped by adding 2 mL of complete media (with 2% FBS). The collagenised tissue fragments were then spinned down thrice at 1000 rpm for 3 minutes, at room temperature in saline buffer. 3 mL complete media (plain DMEM/F-12 media, manufactured by Lonza®) with 2% foetal bovine serum (manufactured by Thermofisher®) along with added epidermal growth factor and insulin (manufactured by Thermofisher®) was added to re-suspend the pellet. The digested tissue fragments were transferred to T25 flask and incubated at 37° C. with 5% CO2.

The flask was incubated for 72 hours without disturbing and the media was replaced every 3 days upto 12^{th} day. On 14^{th} day, the cells were harvested by trypsinization (TrypLE, manufactured by Thermofisher®). This culture P₀ yields epithelial stem cells. The concentration of cells ranges between 4000 to 5000 cells/μL at the time of harvesting. The culture of epithelial stem cells may be cryopreserved for further use.

### Example 2: Harvesting Stromal Stem Cells

The epithelial stem cells (P₀ culture) obtained in the previous example was further differentiated into stromal stem cells. Before the culturing, 1 mL of the spent media was collected into a sterile 1.5 mL microcentrifuge tube for mycoplasma contamination assay.

The media was discarded from the epithelial stem cell culture (P₀ culture) and the cells were washed with phosphate buffer saline (manufactured by Sigma-Aldrich®) The cells were trypsinized by adding 1 mL TrypLE (manufactured by Thermofisher®) The flask was gently tapped 2-3 times and incubated at 37° C. for 2 minutes.

The trypsinized cells were transferred to a 15 mL centrifuge tube containing 1 mL complete media (plain DMEM/F-12 media, manufactured by Lonza®) The flask was washed with 2 mL phosphate buffer saline (manufactured by Sigma-Aldrich®) and added to the centrifuge tube. The cell culture was centrifuged at 1000 rpm for 3 minutes at 25° C. The supernatant was discarded, and the cell pellet was resuspended in 1× phosphate buffer saline.

The resuspended pellet was centrifuged again at 1000 rpm for 3 minutes at 25° C. and resuspended in complete media. 10 μL of 4% Trypan blue stain was mixed with 10 μL, of cell suspension on a strip of parafilm to perform a cell count using Neubauer chamber.

10000×25 cm² cells were taken for plating onto a T25 flask with 2 mL of fresh complete media. The flask was incubated for 72 hours without disturbing and the media was replaced every 3 days upto 12^{th} day. On 14^{th} day, the cells were harvested by trypsinization (TrypLE, manufactured by Thermofisher®). This culture P₁ yields a mixed population of epithelial stem cells and stromal stem cells.

The P₁ culture is sub-cultured for a further generation using the same process as described for obtaining P₂ culture. The P₂ culture has traces of epithelial stem cells and has mostly stromal stem cells.

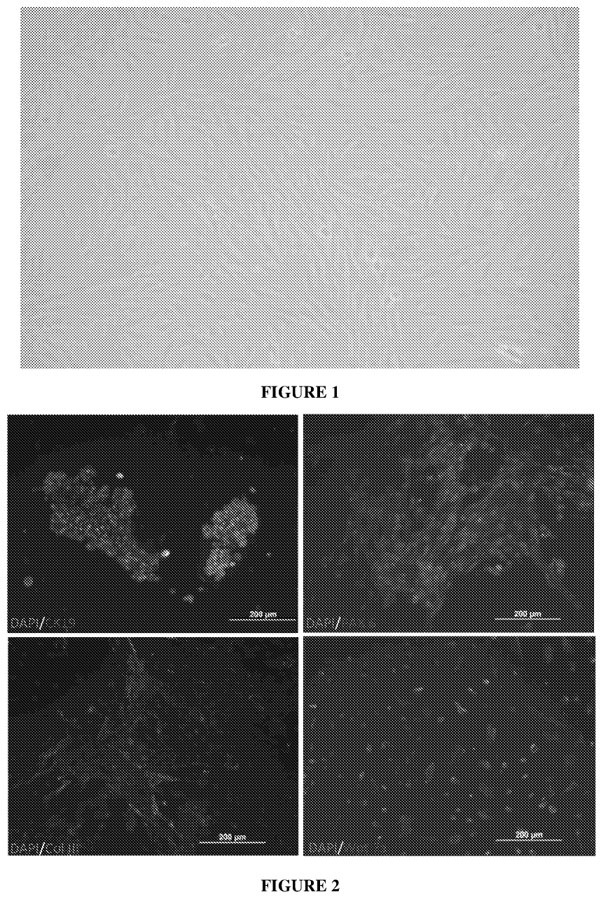

The P₂ culture is sub-cultured for a further generation using the same process as described for obtaining P₃ culture. The P₃ culture yields stromal stem cells (FIG. 1). The concentration of cells ranges between 4000 to 5000 cells/μL at the time of harvesting. The culture of stromal stem cells may be cryopreserved for further use.

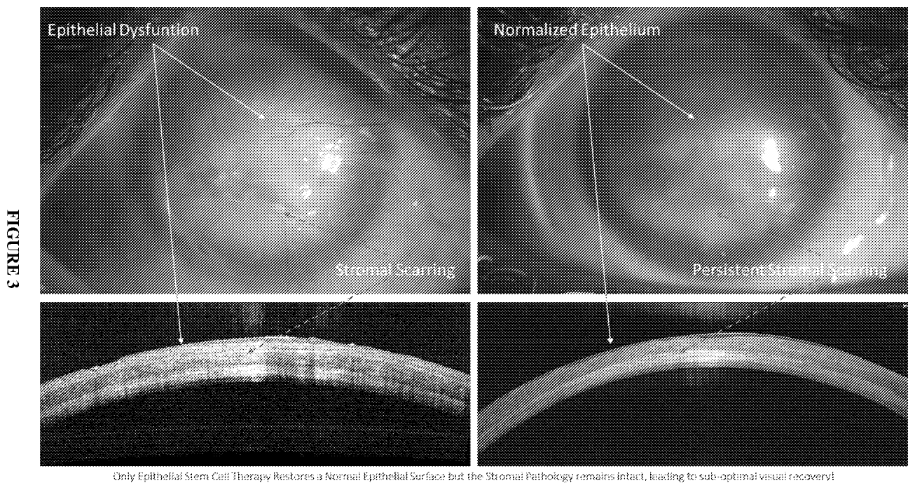

The cultured cells were checked for stemness property and were confirmed for the mesenchymal origin. They expressed the markers CK19, CK3+12, ABCG2, PAX6 and VIMENTIN (FIG. 2) that confirmed their mesenchymal stem cell nature.

### Example 3: Preparation of Cell Composition and Delivery of the Composition to the Patient

The epithelial stem cells and stromal stem cells as harvested from P₀ and P₃ cultures respectively were used for preparation of the multilayer cell composition and delivery to the patient.

The cell cultures were collected and checked for mycoplasma contamination assay.

Further, 10 μL of 4% Trypan blue stain was mixed with 10 μL of each of the cell suspension on a strip of parafilm to perform a cell count using Neubauer chamber to check the viability of the cell culture. The cell count in each of the cell culture must range between 4000 to 5000 cells per μL.

The supernatant of the stromal stem cells was discarded and 100 μL of stromal stem cells were taken. Similarly, supernatant of the epithelial stem cells was discarded and 200 μL of epithelial stem cells were added and mixed with the stromal cells to prepare the cell composition.

The ratio of the stromal stem cells and epithelial stem cells can be customized as per the pathological requirements and the extent of damage to corneal layers. The cell composition of stromal stem cells and epithelial stem cells was delivered to the treatment site of the individual. 40-50 μL of fibrinogen and 40-50 μL (TISSEEL™ Kit) was added for matrix formation and scaffolding.

A canula needle is used for application of the cell composition on the affected part of the cornea. The cornea was covered with contact lens for recovery.

### Example 4: Preparation of Multilayer Composition and Delivery of the Composition to the Patient

The epithelial stem cells and stromal stem cells as harvested from P₀ and P₃ cultures respectively were used for preparation of the multilayer cell composition and delivery to the patient.

The cell cultures were collected and checked for mycoplasma contamination assay.

Further, 10 μL of 4% Trypan blue stain was mixed with 10 μL of each of the cell suspension on a strip of parafilm to perform a cell count using Neubauer chamber to check the viability of the cell culture. The cell count in each of the cell culture must range between 4000 to 5000 cells per μL.

The preparation and delivery of the multi-layer composition to a subject patient was done simultaneously.

The supernatant of the stromal stem cells was discarded and 100 μL of stromal stem cells were taken. 40-50 μL of thrombin (TISSEEL™ Kit) was added to the stromal stem cells. A 1 mL tuberculin syringe was used to mix the stromal cell pellet with the thrombin. A 26X gauze needle was further used for mixing the cells with the thrombin. This composition forms the first layer of the multi-layered composition.

Further, the supernatant of the epithelial stem cells was discarded and 200 μL of epithelial stem cells was taken. 40-50 μL of fibrinogen (TISSEEL™ Kit) was added. A 1 mL tuberculin syringe was used to mix the epithelial cell pellet with the fibrinogen. A 26X gauze needle was further used for mixing the cells with the fibrinogen. This composition forms the second layer of the multi-layered composition.

The ratio of the stromal stem cells and epithelial stem cells can be customized as per the pathological requirements and the extent of damage to corneal layers.

A uniform layer of the stromal cell composition (first layer) was first applied on the affected part of the cornea. Thereafter, a uniform layer of the epithelial cell composition (second layer) was applied on the affected part of the cornea.

A canula needle is used for application of the first and the second layer on the affected part of the cornea. The cornea was covered with contact lens for recovery.

### Example 5: In Vivo Studies

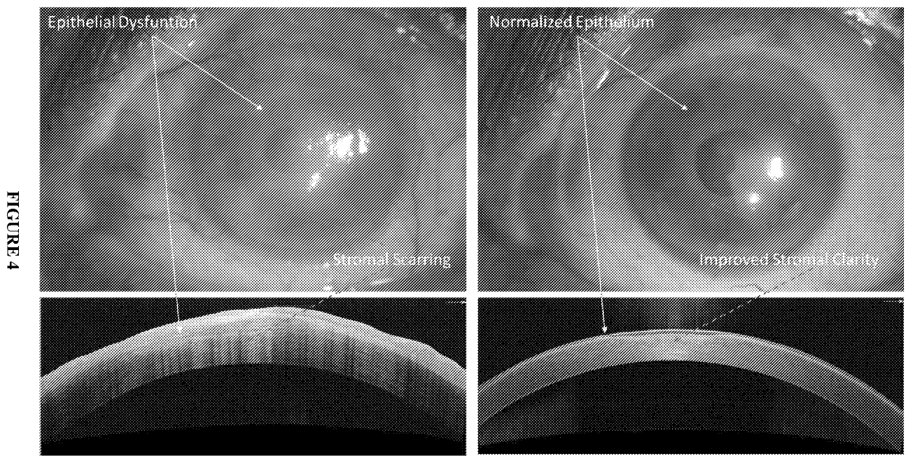

Patients affected by stromal scarring was chosen for the study. In the first group of individuals, about 100 μL epithelial stem cell culture (4000 to 5000 cells/μL) was administered along with 50 μL thrombin and 50 μL fibrinogen (TISSEEL™ Kit). This study represents the state of the art in therapy of corneal epithelial disorder or defect. FIG. 3 depicts the results of epithelial stem cell therapy on a dysfunctional epithelium of a cornea.

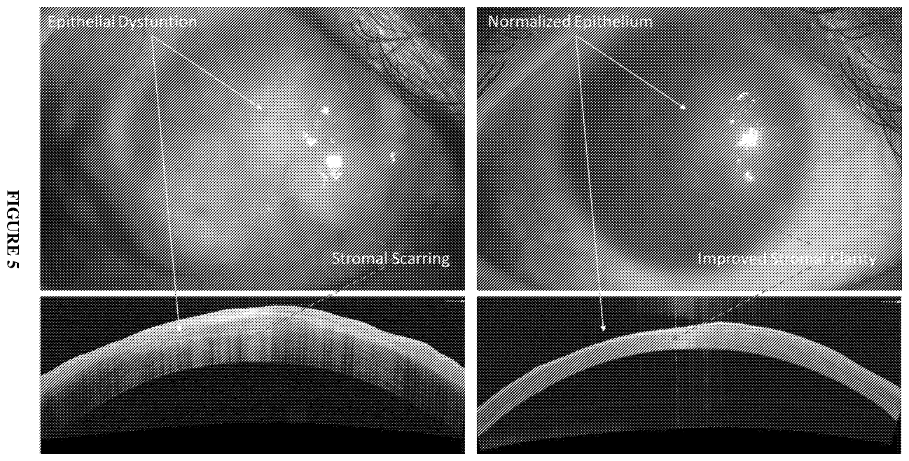

In the second group, the cell composition as described in Example 3 was administered along with 50 μL thrombin and 50 μL fibrinogen (TISSEEL™ Kit). FIG. 4 depicts the results of the application of the cell composition comprising a mixture of epithelial and stromal stem cells on a dysfunctional epithelium of a cornea.

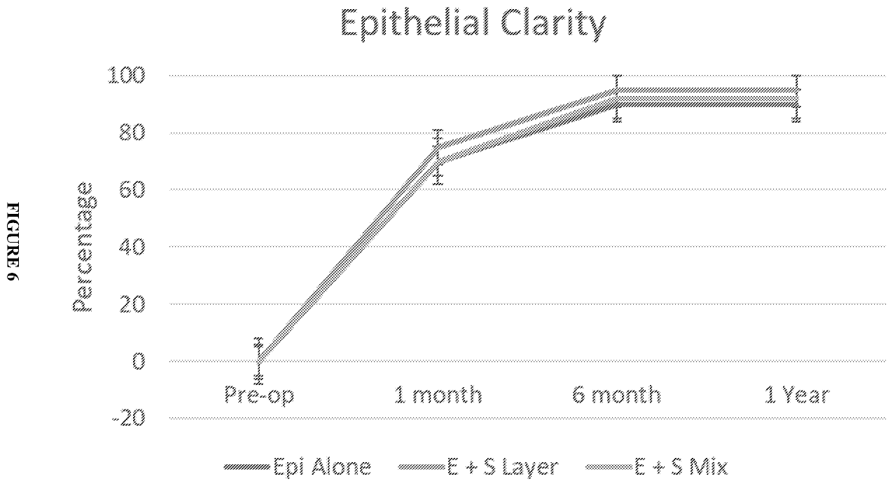

In the third group, the multilayer composition as described in Example 4 was administered. FIG. 5 depicts the results of the application of the multilayer composition on a dysfunctional epithelium of a cornea.

Thereafter, the epithelial clarity, stromal clarity, total corneal clarity and visual recovery was measured in both the groups. The epithelial clarity, stromal clarity and total corneal clarity was measured using digital imaging or corneal densitometry.

TABLE 1
Comparison of the results of epithelial stem cell therapy
and the application of the multilayer composition on
the epithelial clarity of a dysfunctional cornea.

| | | Epithelial clarity (%) | | |
|---|---|---|---|---|
| | | | | Application |
| | | Epithelial Stem | Application of | of multilayer |
| Time | | Cell Therapy | cell composition | composition |
| Pre-operation | | 0 | 0 | 0 |
| 1 | month | 70 | 70 | 75 |
| 6 | months | 90 | 92 | 95 |
| 1 | year | 90 | 92 | 95 |

The results of the studies are depicted in FIG. 6.

TABLE 2
Comparison of the results of epithelial stem cell therapy
and the application of the multilayer composition
on the stromal clarity of a dysfunctional cornea.

| | | Stromal clarity (%) | | |
|---|---|---|---|---|
| | | | | Application |
| | | Epithelial Stem | Application of | of multilayer |
| Time | | Cell Therapy | cell composition | composition |
| Pre-operation | | 35 | 35 | 35 |
| 1 | month | 50 | 75 | 80 |
| 6 | months | 50 | 80 | 85 |
| 1 | year | 50 | 80 | 85 |

The results of the studies are depicted in FIG. 7.

TABLE 3
Comparison of the results of epithelial stem cell therapy
and the application of the multilayer composition on
the total corneal clarity of a dysfunctional cornea.

| | | Total corneal clarity (%) | | |
|---|---|---|---|---|
| | | | | Application |
| | | Epithelial Stem | Application of | of multilayer |
| Time | | Cell Therapy | cell composition | composition |
| Pre-operation | | 15 | 15 | 15 |
| 1 | month | 50 | 65 | 75 |
| 6 | months | 60 | 75 | 85 |
| 1 | year | 65 | 80 | 85 |

The results of the studies are depicted in FIG. 8.

TABLE 4
Comparison of the results of epithelial stem cell therapy
and the application of the multilayer composition
on the visual recovery of a dysfunctional cornea.

| | | Visual recovery (%) | | |
|---|---|---|---|---|
| | | | | Application |
| | | Epithelial Stem | Application of | of multilayer |
| Time | | Cell Therapy | cell composition | composition |
| Pre-operation | | 10 | 10 | 10 |
| 1 | month | 20 | 50 | 65 |
| 6 | months | 40 | 60 | 70 |
| 1 | year | 45 | 65 | 70 |

The results of the studies are depicted in FIG. 9.

It was found that the cell compositions and multilayer compositions shows enhanced efficacious effects with regards to all the parameters, as exhibited in FIGS. 6,7,8 and 9.

It was found that only epithelial stem cell therapy restores a normal epithelial surface, but the stromal pathology remains intact, leading to sub-optimal visual recovery. The cell compositions and multilayer composition restores a normal epithelial surface but also improves stromal clarity, leading to optimal visual recovery.

1. A cell composition comprising a plurality of limbal derived stromal cells and a plurality of limbal derived epithelial cells.

2. The cell composition as claimed in claim 1, wherein the concentration of limbal derived stromal cells and limbal derived epithelial cells ranges from 4000 to 5000 cells/μL.

3. The cell composition as claimed in claim 1, wherein limbal derived stromal cells and limbal derived epithelial cells are present in the ratio from 1:5 to 5:1.

4. The cell composition as claimed in claim 1 further comprising thrombin and fibrinogen, wherein thrombin and fibrinogen are present in the ratio from 1:5 to 5:1.

5. A multilayer cell composition comprising:
a. a first layer comprising a plurality of limbal derived stromal cells and thrombin; and
b. a second layer comprising a plurality of limbal derived epithelial cells and fibrinogen.

6. The multilayer cell composition as claimed in claim 5, wherein the concentration of limbal derived stromal cells and limbal derived epithelial cells ranges from 4000 to 5000 cells/μL.

7. The multilayer cell composition as claimed in claim 5, wherein limbal derived stromal cells and limbal derived epithelial cells are present in the ratio from 1:5 to 5:1.

8. The multilayer cell composition as claimed in claim 5, wherein thrombin and fibrinogen are present in the ratio from 1:5 to 5:1.

9. The cell composition as claimed in any one of claims 1 to 4 or multilayer cell composition as claimed in any one of claims 5 to 8, wherein the limbal derived stromal cells and limbal derived epithelial cells are autologous or allogenic.

10. The cell composition as claimed in any one of claims 1 to 4 or multilayer cell composition as claimed in any one of claims 5 to 8, wherein the cells are obtained from donor tissue or are differentiated from a stem cell in vitro.

11. The cell composition as claimed in any one of claims 1 to 4 or multilayer cell composition as claimed in any one of claims 5 to 8, wherein the composition is layered on the corneal or ocular surface of an individual.

12. A method of preparing a cell composition as claimed in claim 1 comprising the step of mixing a plurality of limbal derived stromal cells and a plurality of limbal derived epithelial cells, wherein the ratio of limbal derived stromal cells to limbal derived epithelial cells ranges from 1:5 to 5:1.

13. The method as claimed in claim 12, wherein the concentration of limbal derived stromal cells and limbal derived epithelial cells ranges from 4000 to 5000 cells/μL.

14. A method of preparing a multilayer cell composition as claimed in claim 5, comprising the steps of:
a. depositing a first layer comprising a plurality of limbal derived stromal cells and thrombin; and
b. depositing a second layer comprising a plurality of limbal derived epithelial cells and fibrinogen, wherein the second layer is deposited over the first layer.

15. The method as claimed in claim 14, wherein the concentration of limbal derived stromal cells and limbal derived epithelial cells ranges from 4000 to 5000 cells/μL.

16. The method as claimed in claim 14, wherein the ratio of limbal derived stromal cells to limbal derived epithelial cells ranges from 1:5 to 5:1.

17. The method as claimed in claim 14, wherein the ratio of thrombin to fibrinogen ranges from 1:5 to 5:1.

18. The method as claimed in any one of claims 12 to 17, wherein the composition is layered on the corneal or ocular surface of an individual.

19. A system for delivering a limbal epithelial tissue producing composition to a treatment site in an individual comprising a delivery system, wherein the delivery system comprises the cell composition as claimed in any one of claims 1 to 4 or multilayer cell composition as claimed in any one of claims 5 to 8.

20. A kit comprising a cell composition as claimed in any one of claims 1 to 4 or multilayer cell composition as claimed in any one of claims 5 to 8 and a delivery system for delivering the cell composition to a treatment site in an individual.

21. A method of treating a disease or disorder of the eye in a patient, comprising the step of layering a cell composition as claimed in any one of claims 1 to 4 or multilayer cell composition as claimed in any one of claims 5 to 8 on the corneal or ocular surface of an individual.

22. The method as claimed in claim 21, wherein said disease or disorder of the eye is a non-healing corneal epithelial defect or persistent corneal epithelial defect.

23. Use of a cell composition as claimed in any one of claims 1 to 4 or multilayer cell composition as claimed in any one of claims 5 to 8 for treating a disease or disorder of the eye in a patient, wherein said disease or disorder of the eye is a non-healing corneal epithelial defect or persistent corneal epithelial defect.
